(19) United States
(12) Patent Application Publication
(10) Pub. No.: US 2012/0265169 A1
(43) Pub. Date: Oct. 18, 2012

(54) SYSTEMS AND METHODS FOR COLLECTING, STORING, AND ADMINISTERING FLUID FROM A BREAST

(76) Inventors: Jules Sherman, Palo Alto, CA (US); Lital Yizhar, Rehovot (IL)

(21) Appl. No.: 13/447,186

(22) Filed: Apr. 14, 2012

Related U.S. Application Data
(60) Provisional application No. 61/475,578, filed on Apr. 14, 2011.

(51) Int. Cl. A61M 1/06

(52) U.S. Cl. 604/514; 604/74

(57) ABSTRACT

A device for collecting and administering fluid from a breast includes a container having a first end with a first opening for collecting fluid from the breast, and a second end with a second opening for administering the collected fluid, the second opening being smaller than the first opening, and a coupler for detachably coupling to the first end of the container, wherein the coupler comprises threads on an exterior surface for detachably coupling to a pump connector.

### CROSS-REFERENCE TO RELATED APPLICATION

This application claims the benefit of U.S. Provisional Application No. 61/475,578 filed on Apr. 14, 2011, which is hereby incorporated by reference in its entirety for all purposes.

### FIELD

The present application pertains generally to systems and methods for collecting and administering fluid, such as colostrum and milk, from a breast of a mammalian body.

### BACKGROUND

Colostrum is fluid produced by the mammary glands of mammals in late stage of pregnancy, such as just prior to giving birth, and sometimes even after pregnancy. Colostrum has higher protein content than ordinary milk, and contains antibodies to protect the newborn against disease. Colostrum is essentially newborn's first food, and helps the newborn transition to consumption of mature milk. Thus, it is important that colostrum be fed to newborn after birth.

In the case of humans, in certain situations, the newborn may be separated from the mother immediately after birth. For example, the newborn may need to be treated right after birth, such as due to premature birth, infection, or other medical conditions. In such cases, the mother would not be able to directly feed the newborn from her breasts. Other case, such as maternal pain, infant inability to effectively suck, etc., often prevent direct breast feeding in the early post partum stage. Also, a mother may choose not to directly breastfeed the newborn, and elect to use a device for administering the colostrum to the newborn.

Various techniques may be used to provide colostrum to newborn. For example, a medicine cup or a plastic spoon may be used to collect the colostrum from the mother's breast(s). In some cases, the collected colostrum is then transferred to a syringe, which is then used to administer the colostrum to the newborn. In other cases, the colostrum is administered to the baby directly from the cup/spoon.

Applicant of the subject application determines that it would be desirable to provide a system and method for collecting, storing, and administering breast fluid, such as colostrum or milk, to a subject.

### SUMMARY

In accordance with some embodiments, a device for collecting and administering fluid from a breast includes a container having a first end with a first opening for collecting fluid from the breast, and a second end with a second opening for administering the collected fluid, the second opening being smaller than the first opening, and a coupler for detachably coupling to the first end of the container, wherein the coupler comprises threads on an exterior surface for detachably coupling to a pump connector.

In accordance with other embodiments, a device for collecting and administering fluid from a breast includes a container having a first end with a first opening for collecting fluid from the breast, and a second end with a second opening for administering the collected fluid, the second opening being smaller than the first opening, and a plunger configured to detachably couple to the first end of the container, wherein the container comprises a lip at the first end that circumferentially surrounds the first opening.

In accordance with some embodiments, a method for collecting and administering fluid from a breast includes detachably coupling a pump connector to a container, the pump connector being a component of a pump system, wherein the container has a first end with a first opening, and a second end with a second opening, using the pump system to collect fluid from the breast, and using the container to receive the fluid through the first opening at the first end.

Other and further aspects and features will be evident from reading the following detailed description of the embodiments.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 is a perspective view of a device for collecting and administering colostrum/milk in accordance with some embodiments.

FIG. 2 is a side view of the device of FIG. 1.

FIG. 3 is a side cross sectional view of the device of FIG. 1.

FIG. 4 is an exploded view of the device of FIG. 1, particularly showing the device being used with a pump.

FIG. 5 illustrates the container of FIG. 1, particularly showing the container having multiple markings in accordance with some embodiments.

FIG. 6 illustrates the device of FIG. 1, particularly showing the container with the coupler and plunger removed.

FIG. 7 illustrates the device of FIG. 6, particularly showing the container with a lip coupled thereto.

FIG. 8 illustrates another device for collecting and administering colostrum/milk in accordance with other embodiments.

### DETAILED DESCRIPTION

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

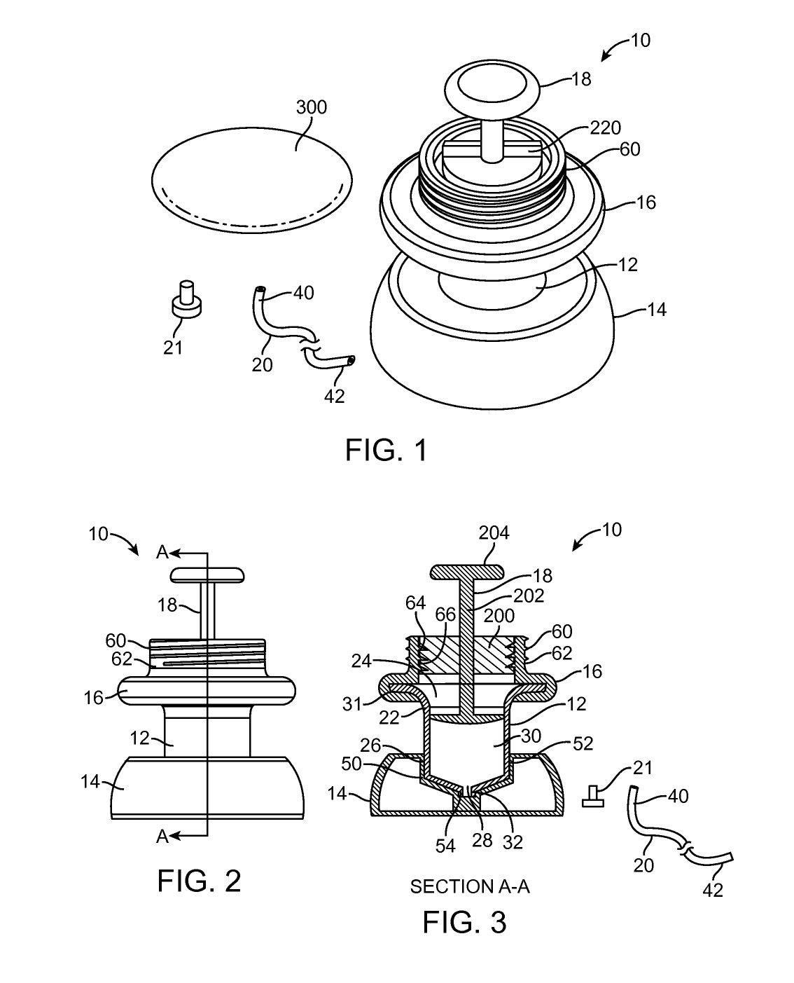

FIG. 1 is a perspective view of a device 10 for collecting and administering colostrum in accordance with some embodiments. FIG. 2 is a side view of the device 10, and FIG. 3 is a side cross sectional view of the device 10. The device 10 will be described with reference to collecting and administering colostrum from a breast. However, in other embodiments, the device 10 may be used to collect and administering mature milk from a breast. Thus, as used in this specification, the term “fluid” is not limited to colostrum, and may refer to mature milk as well.

As shown in FIGS. 1-3, the device 10 includes a container 12, a base 14, a coupler 16, a plunger 18, a tube 20, a blocker 21, and a lid 300. The container 12 has a first end 22 with a first opening 24, a second end 26 with a second opening 28, and a compartment 30 extending from the first end 22 to the second end 26. The first opening 24 is configured to receive colostrum from a breast, the compartment 30 is configured to contain the received colostrum, and the second opening 28 is configured to deliver the colostrum from the compartment 30 to a subject (e.g., an infant, toddler, etc.).

As shown in FIG. 3, the first end 22 of the container 12 has a lip 31 that surrounds the first opening 24. The lip 31 (and optionally also the rest of the container 12) may be made from a hydrophobic material, which allows fluid to slid along the surface of the lip 31 (and the container 12). The lip 31 extends away from an axis of the container 12 in a flare-configuration, and has a curvilinear profile. Such configuration is advantageous because it allows the container 12 to be placed directly against a breast to collect breast fluid using hand expression, wherein the lip 31 prevents or reduces the risk of spillage, and the curvature of the lip 31 allows the breast fluid to “slide” to the bottom of the container 12. Furthermore, the lip 31 provides a ledge that allows a user to hold the container 12 by placing fingers underneath it. In addition, the lip 31 may have a profile that is designed for efficient cup feeding. In other embodiments, the lip 31 may have other configurations. For example, in other embodiments, the lip 31 may have a funnel shape, and/or a rectilinear configuration. Also, in other embodiments, the lip 31 is optional, and the container 12 may not include any lip at the first end 22 of the container 12.

In the illustrated embodiments, the second end 26 of the container 12 has a protrusion 32 that defines the second opening 28. During use, when the container 12 is used to collect colostrum from a breast, the blocker 21 may be detachably coupled to the protrusion 32 that that it covers the second opening 28. For example, the blocker 21 may be configured (e.g., sized and/or shaped) to be inserted into the second opening 28 so that the blocker 21 is frictionally engaged with an interior surface of the protrusion 32. Alternatively, the blocker 21 may be configured (e.g., sized and/or shaped) to be placed around the protrusion 32 so that the blocker 21 frictionally engages with an exterior surface of the protrusion 32. When the blocker 21 is detachably coupled to the second end 26 of the container 12, it prevents fluid in the container 12 from exiting through the second opening 28 at the second end 26 of the container 12. It should be noted that the manner in which the blocker 21 is detachably couple to the container 12 is not limited to the example discussed previously, and that blocker 21 may be detachably coupled to the container 12 in other configurations. For example, in other embodiments, the blocker 21 may include a connector for detachably coupling to the second end 26 of the container 12. For example, the connector may include threads that are configured to threadingly engage with corresponding threads at the protrusion 32 (either at an exterior surface of the protrusion 32, or an interior surface of the protrusion 32) of the container 12. Furthermore, in other embodiments, the protrusion 32 is optional, and the container 12 may not include the protrusion 32. In such cases, the blocker 21 may be coupled to the container 12 by inserting the blocker 21 into the second opening 28 at a bottom surface of the container 12. Also, in other embodiments, the device 10 may further include a member (not shown) that connects the blocker 21 to the container 12. The member may have one end connected to the blocker 21, and another end connected to the container. The member prevents the blocker 21 from being separated from the container 12 even when the blocker 21 is not inserted into the second opening 28, thereby preventing the blocker 21 from getting lost.

After fluid has been collected inside the container 12, the blocker 21 may be removed from the container 12, and a first end 40 of the tube 20 may be detachably coupled to the second end 26 of the container 12 by inserting the first end 40 of the tube 20 into the second opening 28, so that the tube 20 is detachably coupled to the container 12 using friction. When the tube 20 is coupled to the container 12, a lumen in the tube 20 is in fluid communication with the second opening 28. A second end 42 of the tube 20 may be placed into a mouth of the subject. The tube 20 then functions as a feeding tube for delivering colostrum from the container 12 to the subject. In some embodiments, the tube 20 may be a 5 French feeding tube. In other embodiments, the tube 20 may have other dimensions.

In other embodiments, the tube 20 is optional, and the device 10 may not include the tube 20. In such cases, the protrusion 32 at the second end 26 of the container 12 may be placed directly into a mouth of the subject for administering the colostrum to the subject. It should be noted that the manner in which the tube 20 is detachably couple to the container 12 is not limited to the example discussed previously, and that tube 20 may be detachably coupled to the container 12 in other configurations. For example, in other embodiments, the tube 20 may be configured (e.g., sized and/or shaped) so that it is placed around the protrusion 32 at the second end 26 of the container 12, and is detachably secured to the container using friction. Also, in other embodiments, the first end 40 of the tube 20 may include a connector for detachably coupling to the second end 26 of the container 12. For example, the connector may include threads that are configured to threadingly engage with corresponding threads at the protrusion 32 (either at an exterior surface of the protrusion 32, or an interior surface of the protrusion 32) of the container 12. Furthermore, in other embodiments, the protrusion 32 is optional, and the container 12 may not include the protrusion 32. In such cases, the first end 40 of the tube 20 may be coupled to the container 12 by inserting the first end 40 into the second opening 28 at the bottom surface of the container 12.

As shown in FIG. 3, the base 14 has a recess 50 with a first recess portion 52 configured (e.g., sized and/or shaped) to accommodate the second end 26 of the container 12, and a second recess portion 54 that is smaller than the first recess portion 52. The second recess portion 54 is configured to accommodate the protrusion 32 of the container 12. The base 14 allows the container 12 to be supported in a vertical position when the container 12 is not being used.

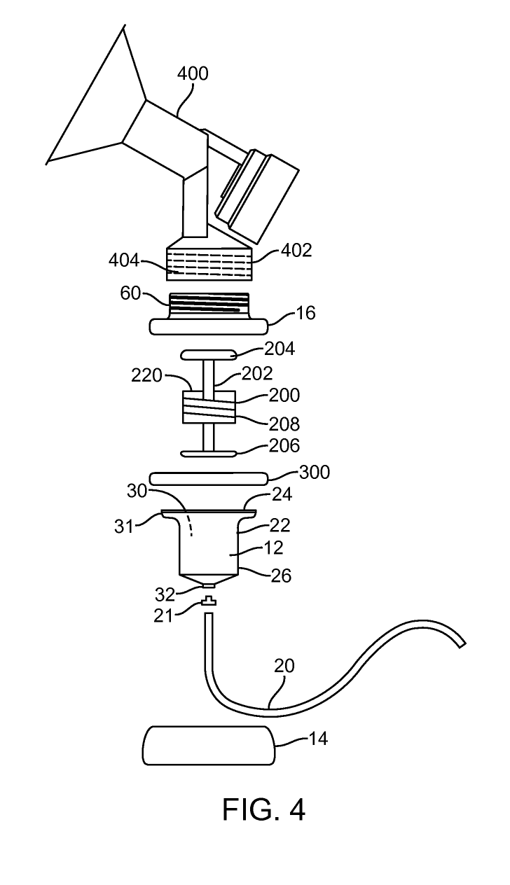

In the illustrated embodiments, the coupler 16 has a ring configuration, and includes threads 60 at an exterior surface 62 of the coupler 16. The coupler 16 is configured to detachably couple to the first end 22 of the container 12 during use. As shown in the illustrated embodiments, the coupler 16 has a latch that extends circumferentially around the coupler 16, which allows the coupler 16 to detachably couple to the lip 31 of the container by a snap-fit connection. When the coupler 16 is coupled to the container 12, the coupler 16 may be used to detachably couple to a pump connector of a pump system, so that the pump system may be used to collect fluid (e.g., colostrum, milk) from a breast, and deliver the fluid into the container 12. FIG. 4 illustrates a suction cup 400 which is a part of the pump system (not shown) that is configured to provide suction through the suction cup (pump flange) 400 to express or extract fluid from within the breast, wherein the pump system may be a manually actuated pump or an electric pump. In some cases, the suction cup 400 may be any of the breastshield (pump flange) manufactured by Medela®, Ameda®, Evenflow®, or other manufacturers. In other embodiments, the suction cup 400 may be provided by other manufacturers, or may be provided by a manufacturer of the device 10. Also, in some embodiments, the suction cup 400 may be considered to be a part of the device 10. The suction cup 400 includes a connector 402 with threads 404 that threadingly engage with the threads 60 at the exterior surface 62 of the coupler 16. In the case in which the suction cup 400 is provided by a different manufacturer from that of the device 10, wherein the suction cup 400 is configured to detachably couple to a baby bottle, the coupler 16 may be configured so that it can mate with the connector 402 of the suction cup 400, thereby allowing the container 12 to be used in place of the baby bottle. In particular, the coupler 16 may have an exterior dimension that allows the portion with the threads 60 to be inserted into the connector 402, and the threads 60 may have a pitch and dimension that allows the threads 60 to fittingly engage with the threads 404 at the connector 402. The coupler 16 may be made from silicone, polypropylene, other suitable materials, or combination thereof, in different embodiments. Also, in some cases, different parts of the coupler 16 may be made from different materials having different stiffnesses. For example, in some embodiments, the top part of the coupler 16 may be made from a first material (e.g., polypropylene) that is stiffer than a second material (e.g., silicone) for the bottom part of the coupler 16.

As shown in FIGS. 3 and 4, the plunger 18 has a coupler 200, a shaft 202 extending through the coupler 200, a manual actuator 204 attached to one end of the shaft 202, and a plunger head 206 attached to another end of the shaft 202. The coupler 200 includes two or more tabs 220, which allows a user to turn the coupler 200. The shaft 202 is slidably coupled to the coupler 200 so that the plunger head 206 may be advanced towards a base of the container 12 by pressing the actuator 204 relative to the coupler 200, or may be retracted away from the base of the container 12 by pulling the actuator 204 relative to the coupler 200. As shown in the illustrated embodiments, the coupler 200 of the plunger 18 has threads 208 at an exterior surface of the coupler 200. The threads 208 of the coupler 200 are configured to mate with threads 64 at an interior surface 66 of the coupler 16.

In other embodiments, instead of providing the tabs 220 for turning the coupler 200, the plunger 18 may have other mechanisms for turning the coupler 200. For example, in other embodiments, the shaft 202 of the plunger 18 may have a groove that slidingly mates with a protrusion at an interior surface of the coupler 200. Such configuration allows the shaft 202 to slidingly move relative to the coupler 200 along a longitudinal axis of the shaft 202, while also allowing the coupler 200 to be turned about the longitudinal axis of the shaft 202 by turning the actuator 204 about the longitudinal axis of the shaft 202.

During use, the plunger 18 may be detachably coupled to the container 12. In particular, the coupler 16 may be used to couple the plunger 18 to the container 12 by detachably securing the lip 31 of the container 12 to the circumferential latch of the coupler 16 via the snap-fit connection, and by detachably securing the plunger 18 to the top portion of the coupler 16 using the threads 208 and 64 (i.e., by turning the coupler 200 of the plunger 18 relative to the coupler 16 using the tabs 220). The actuator 204 may then be used to advance the plunger head 206 inside the compartment 30 of the container 12 to push fluid out of the second opening 28 at the second end 26 of the container 12.

It should be noted that the manner in which the plunger 18 is coupled to the container 12 is not limited to the embodiments described. In other embodiments, the plunger 18 may be directly or indirectly coupled to the container 12 using other mechanisms. For example, in other embodiments, the coupler 200 of the plunger 18 may be screwed into the container 12 having corresponding threads at an interior surface of the container 12 at the first end 22. In further embodiments, the coupler 200 of the plunger 18 may not have threads 60. Instead, the coupler 200 of the plunger 18 may be configured to secure itself directly to the container 12 using a snap-fit connection, a frictional engagement, or any of other types of connection. In still further embodiments, the coupler 200 of the plunger 18 may be configured to secure itself directly to the coupler 16 (and indirectly to the container 12) using a snap-fit connection, a frictional engagement, or other types of connection.

As shown in FIGS. 1, 4, and 7, the lid 300 of the device 10 is configured for covering the first opening 24 of the container 12. The lid may be made from silicone, a polymer, or other suitable material(s). The lid 300 is useful when the coupler 16 and the plunger 18 are not coupled to the container 12, and when the user wishes to transport the collected fluid in the container 12. In such cases, the lid 300 may be detachably coupled to the first end 22 of the container 12. The lid 300 includes a latch that extends circumferentially around the lid 300 (as similarly discussed with reference to the coupler 16). The latch allows the lid 300 to detachably couple to the lip 31 of the container 12 using a snap-fit connection. In the illustrated embodiments, the lid 300 is configured (e.g., sized, shaped, etc.) to detachably couple to the container 12. In other embodiments, the lid 300 may be configured to detachably couple to the coupler 16. Also, in other embodiments, the lid 300 may be configured to detachably couple to the container 12/coupler 16 using other mechanisms other than a snap-fit mechanism. For example, in other embodiments, the lid 300 may be configured to detachably couple to the container 12/coupler 16 using screw threads, clip(s), friction, etc.

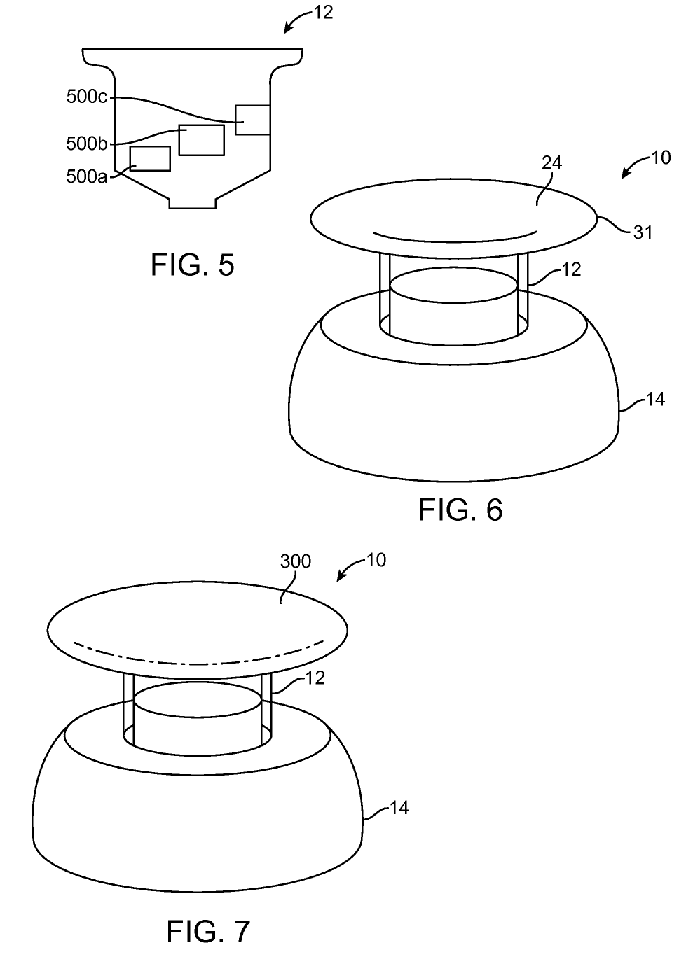

In any of the embodiments described herein, the container 12 may include one or more markings for indicating target fluid level(s). FIG. 5 illustrates the container 12, particularly showing the container 12 having multiple markings 500a-500c in accordance with some embodiments. The first marking 500a indicates a first range (e.g., 2-10 ml) of desired fluid volume in the first day (day 1) after childbirth, the second marking 500b indicates a second range (e.g., 5-15 ml) of desired fluid volume in the second day (day 2) after childbirth, and the third marking 500c indicates a third range (e.g., 15-30 ml) of desired fluid volume in the third day (day 3) after childbirth. The ranges are illustrated as having overlapping regions. In other embodiments, two adjacent ranges may not be overlapping. Also, in further embodiments, the three ranges of volumes may be different from the examples mentioned. In some embodiments, the markings 500a-500c may be molded during the manufacturing of the container 12. In other embodiments, the markings 500a-500c on the container 12 may be color coded, wherein different colors represent different respective ranges of target fluid volume. The markings 500a-500c are advantageous because it allows a user of the device 10 to gage how much fluid should be collected from the breast.

In the above embodiments, the markings 500a-500c represent different respective ranges of target volume. In other embodiments, the markings 500a-500c may represent different target volume levels (i.e., instead of ranges of levels). Also, in the above embodiments, the markings 500a-500c indicate different respective ranges of fluid volume desired to be collected in different days after childbirth. In other embodiments, instead of “day(s)” after childbirth, the markings 500a-500c may correspond with different hour(s) after birth, number of meals, or other units that correspond with passage of time. In further embodiments, instead of three markings 500a-500c, the container 12 may include less than three markings or more than three markings. Also, in other embodiments, instead of, or in addition to, the configuration shown, the markings 500a-500c may include numerical information on the container 12, such as volume levels (e.g., in unit of ml, cc, etc.), “1” in the region for marking 500a, “2” in the region for marking 500b, and “3” in the region for marking 500c.

In any of the embodiments described herein, any of the components of the device 10 may be made from a suitable polymer, plastic, silicone, other materials, or combination thereof. In some cases, any components of the device 10 (e.g., the container 12, plunger 18, lid 300) that may potentially come into contact with breast fluid may be made from a BPA-free material.

Also, in any of the embodiments, the container 12 may have a size that is smaller than a size of a baby bottle. For example, in any of the embodiments described herein, the container 12 may have a total height that is anywhere between 0.5 inch and 3 inches, and more preferably, between 1 inch and 2 inches (e.g., 1.5 inch). Also, in some embodiments, the container 12 may be configured to hold at most 40 ml of fluid, and more preferably, at most 30 ml of fluid, or less. Such size feature is advantageous because the amount of colostrum produced by a mother may not be significant, and if a large container (e.g., a baby bottle) is used to collect the colostrum, a mother may be discouraged from breastfeeding when she sees that there is very little colostrum relative to the size of the container. Thus, providing a container 12 that is smaller than a size of a baby bottle has functional reasons, and is not a matter of design choice. FIG. 3 provides some exemplary dimensions (in unit of inch) for the device 10 in accordance with some embodiments. In other embodiments, the container 12 may have other dimensions that are different from those described.

A method of using the device 10 in accordance with some embodiments will now be described. First, to set up the device 10 for collecting breast fluid (e.g., colostrum, milk), the plunger 18 is separated from the container 12 by turning the coupler 200 of the plunger 18 relative to the coupler 16 (e.g., using the tabs 220) until the threads 208 and 64 are decoupled from each other. The coupler 16 is detachably coupled to the first end 22 of the container 12 by snap-fitting the edge of the coupler 16 against the lip 31 of the container 12, and the suction cup 400 of a pump system is detachably coupled to the coupler 16 by screwing the coupler 16 relative to the connector 402 of the suction cup 400. Thus, the suction cup 400 is secured to the container 12 through the coupler 16. The blocker 21 is then used to cover the second opening 28 at the second end 26 of the container 12. The suction cup 400 is placed over the breast, and the pump system is then activated to create suction at the suction cup 400, thereby expressing or extracting breast fluid out of the holes in the nipple at the breast. The collected breast fluid enters the container 12 through the first opening 24 at the first end 22 of the container 12, and is contained in the compartment 30 of the container 12.

In other embodiments, instead of using a pump system to collect the colostrum/milk, hand expression may be employed to collect the colostrum/milk. In such cases, the coupler 16 may be removed from the container 12 (FIG. 6). The first end 22 of the container 12 is then placed below a nipple, so that fluid exiting from the nipple resulted from hand expression may be collected by the container 12.

After the breast fluid has been collected, the suction cup 400 is detached from the coupler 16. If hand expression was used to collect the breast fluid, the coupler 16 is then detachably coupled to the container 12. The plunger 18 is then detachably coupled to the first end 22 of the container 12 through the coupler 16. In particular, the plunger 18 is inserted into the top portion of the coupler 16, and is turned using the tabs 220 to threadingly engage the threads 208 at the plunger 18 against the threads 64 at the interior surface 66 of the coupler 16. After the plunger 18 is mounted, the plunger head 206 functions as a seal to prevent the collected breast fluid in the container 12 from leaking out of the first opening 24 of the container 12. With both the first and second openings 24, 28 covered and sealed, the container 12 may then be used to transport the collected breast fluid from the mother to a subject.

Alternatively, instead of using the plunger 18 to seal the first end 22 of the container 12, the lid 300 may be used to cover the first opening 24 at the first end 22 of the container 12. In such cases, the coupler 16 is not coupled to the container. Instead, the lid 300 is snap-fit against the lip 31 of the container 12 to thereby seal the first opening 24 at the container 12 (FIG. 7). The container 12 may then be used to transport the collected breast fluid from the mother to a subject.

In some cases, when a nurse or a personnel at a hospital is not ready to transport the collected breast fluid, or when the mother wishes to collect more breast fluid before the container 12 is delivered to the subject, the second end 26 of the container 12 may be placed into the recess 50 of the base 14, and the container 12 may be vertically supported by the base 14.

Once delivered to the subject, the container 12 with the breast fluid may then be used to feed the subject. If the plunger 18 is already coupled to the container 12, the container 12 may then be turned upside down, and the blocker 21 is then removed from the second end 26 of the container 12. On the other hand, if the plunger 18 is not already coupled to the container 12 (i.e., if the lid 300 is on the container), the lid 300 may first be removed from the container 12, and then the coupler 16 and the plunger 18 may be coupled to the container 12.

In some embodiments, the protrusion 32 at the second end 26 of the container 12 may be placed into the subject's mouth. The user of the device 10 may then advance the plunger head 206 inside the container 12 by pressing on the actuator 204 of the plunger 18, thereby pushing the breast fluid out of the second opening 28 and into the subject's mouth. In other embodiments, the first end 40 of the feeding tube 20 may be detachably coupled to the second end 26 of the container 12, and the second end 42 of the feeding tube 20 may be placed inside the subject's mouth. The user of the device 10 may then advance the plunger head 206 inside the container 12 by pressing on the actuator 204 of the plunger 18, thereby pushing the breast fluid out of the second opening 28, into the feeding tube 20, and into the subject's mouth.

When the feeding is completed, or if the person feeding the subject wishes to temporarily stop the feeding, the second opening 28 of the container 12 may be closed by using the blocker 21, and the container 12 may be vertically supported by the base 14. In some embodiments, the device 10 includes one base 14, in which cases, when the container 12 is being delivered from the mother to the subject, the base 14 may also be delivered together with the container 12 so that the person feeding the subject may use the base 14 to support the container 12 if desired. In other embodiments, the device 10 may include two bases 14. In such cases, one base 14 may stay with the mother so that she can use the base 14 to support the container 12, and the other base 14 may stay with a personnel at the hospital so that the personnel feeding the subject can use the base 14 to support the container 12.

In some embodiments, all of the components of the device 10 are for a single use. In other embodiments, any one or more of the components (such as the base 14) of the device 10 may be for multiple use.

As illustrated in the above embodiments, the device 10 is advantageous because the same device 10 may be used for collecting colostrum/milk, storing the colostrum/milk, transporting the colostrum/milk, and administering the colostrum/milk to a subject. Thus, the device 10 obviates the need to transfer colostrum/milk from a medicine cup, spoon, pump bottle, or other containers, to another container for administering of the fluid to the subject. The device 10 is also advantageous because it provides two modes of breast fluid collection, wherein in the first mode, the device 10 is detachably coupled to a pump system to receive breast fluid from the pump system, and in the second mode, the device 10 is not detachably coupled to any pump system, but is placed directly next to a breast for collecting breast fluid resulted from hand expression. The device 10 promotes feeding the newborn with human colostrums/milk, which in turn may result in lower health care costs, and many other benefits.

Although embodiments of the device 10 have been described with reference to the device 10 having the coupler 16, in other embodiments, the coupler 16 is optional, and the device 10 may not include the coupler 16. In such cases, instead of using a pump system to collect colostrum/milk into the container 12, the colostrum/milk may be collected into the container 12 manually by using hand expression. After the breast fluid has been collected into the container 12, the plunger 18 may then be inserted into the compartment 30 of the container 12 to push the fluid out of the second opening 28 of the container 12.

In still further embodiments, the device 10 may include the coupler 16, but the coupler does not include the threads 60 or any other mechanism for coupling to a pump connector. In such cases, instead of using a pump system to collect colostrum/milk into the container 12, the colostrum/milk may be collected into the container 12 manually by using hand expression. After the breast fluid has been collected into the container 12, the coupler 16 may then be used to detachably couple the plunger 18 to the first end 22 of the container, as similarly discussed. The plunger 18 may then be used to push the fluid out of the second opening 28 of the container 12.

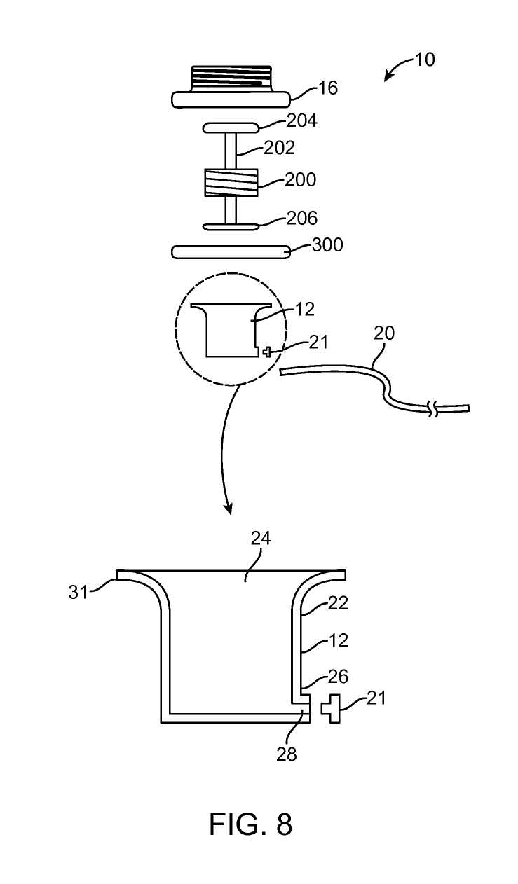

In the above embodiments, the second opening 28 has been illustrated as being at the bottom surface of the container 12. In other embodiments, the second opening 28 may be implemented at other locations at the container 12. For example, in other embodiments, the second opening 28 may be implemented at a side of the container 12 (FIG. 8). Such configuration obviates the need to have the base 14, and the container 12 may be directly placed on a flat surface. Also, in further embodiments, instead of having a protrusion that sticks out from a side of the container 12, as illustrated in the figure, the container 12 may not have the protrusion. In such cases, the second opening 28 may be implemented at a side wall of the container 12.

Also, in further embodiments, the plunger 18 may be at other locations. For example in other embodiments, the container 12 may include a side opening at the side wall of the container 12 that is configured to allow an insertion of the plunger 18. In such cases, while the first opening 24 of the container 12 is being used to collect colostrum/milk, the plunger 18 may remain coupled to the side of the container 12. After the colostrum/milk is collected into the container 12, the lid 300 may be used to cover the first opening 24, or the top of the coupler 16 (if the coupler 16 is already coupled to the container 12). The blocker 21 is then removed from the second opening 28 of the container 12. The plunger 18 at the side of the container 12 may then be actuated to push the collected colostrum/milk out of the second opening 28, either directly from the second opening 28 into a subject's mouth, or from the second opening 28 into a feeding tube.

Although particular embodiments have been shown and described, it will be understood that they are not intended to limit the claimed inventions, and it will be obvious to those skilled in the art that various changes and modifications may be made without departing from the spirit and scope of the claimed inventions. The claimed inventions are intended to cover alternatives, modifications, and equivalents.

What is claimed:

1. A device for collecting and administering fluid from a breast, comprising:
a container having a first end with a first opening for collecting fluid from the breast, and a second end with a second opening for administering the collected fluid, the second opening being smaller than the first opening; and
a coupler for detachably coupling to the first end of the container, wherein the coupler comprises threads on an exterior surface for detachably coupling to a pump connector.

2. The device of claim 1, further comprising a plunger configured to detachably couple to the first end of the container.

3. The device of claim 2, wherein the plunger is configured to indirectly detachably couple to the first end of the container through the coupler.

4. The device of claim 1, further comprising a tube configured to detachably couple to the second end of the container, wherein when the tube is detachably coupled to the second end of the container, a lumen in the tube is in fluid communication with the second opening.

5. The device of claim 1, further comprising a blocker configured for detachably coupling to the second end of the container to cover the second opening.

6. The device of claim 5, further comprising a tube for detachably coupling to the second end of the container to fluidly communicate with the second opening when the blocker is detached from the second end.

7. The device of claim 1, wherein the first end of the container comprises a lip that circumferentially surrounds the first opening.

8. The device of claim 1, further comprising a base with a recess for accommodating the second end of the container.

9. The device of claim 8, wherein the recess has a first portion for accommodating the second end of the container, and a second portion that is smaller than the first portion.

10. The device of claim 1, further comprising a first marking on the container, wherein the first marking is configured to indicate a first target fluid range.

11. The device of claim 10, further comprising a second marking on the container, wherein the second marking is configured to indicate a second target fluid range, wherein the first target fluid range indicates a first range of desired amount of fluid to be achieved at a first time period, and the second target fluid range indicates a second range of desired amount of fluid to be achieved at a second time period.

12. The device of claim 1, further comprising a lid for covering the first opening, wherein the lid is configured for detachably coupling to the first end of the container when the coupler is not coupled to the first end of the container.

13. The device of claim 1, further comprising a lid configured for detachably coupling to the coupler to thereby cover the coupler.

14. The device of claim 1, wherein the container has a volume capacity that is less than 40 ml.

15. A device for collecting and administering fluid from a breast, comprising:
a container having a first end with a first opening for collecting fluid from the breast, and a second end with a second opening for administering the collected fluid, the second opening being smaller than the first opening; and
a plunger configured to detachably couple to the first end of the container;
wherein the container comprises a lip at the first end that circumferentially surrounds the first opening.

16. The device of claim 15, further comprising a coupler for detachably coupling to the first end of the container, wherein the coupler comprises threads on an exterior surface for detachably coupling to a pump connector.

17. The device of claim 16, further comprising a lid for covering the first opening, wherein the lid is configured for detachably coupling to the first end of the container when the coupler is not coupled to the first end of the container.

18. The device of claim 15, further comprising a tube configured to detachably couple to the second end of the container, wherein when the tube is detachably coupled to the second end of the container, a lumen in the tube is in fluid communication with the second opening.

19. The device of claim 15, further comprising a blocker configured for detachably coupling to the second end of the container to cover the second opening.

20. The device of claim 19, further comprising a tube for detachably coupling to the second end of the container to fluidly communicate with the second opening when the blocker is detached from the second end.

21. The device of claim 15, further comprising a base with a recess for accommodating the second end of the container.

22. The device of claim 21, wherein the recess has a first portion for accommodating the second end of the container, and a second portion that is smaller than the first portion.

23. The device of claim 15, further comprising a first marking on the container, wherein the first marking is configured to indicate a first target fluid range.

24. The device of claim 23, further comprising a second marking on the container, wherein the second marking is configured to indicate a second target fluid range, wherein the first target fluid range indicates a first range of desired amount of fluid to be achieved at a first time period, and the second target fluid range indicates a second range of desired amount of fluid to be achieved at a second time period.

25. The device of claim 15, further comprising a coupler, wherein the plunger is configured to detachably couple to the first end of the container indirectly using the coupler.

26. The device of claim 25, wherein the plunger comprises a coupling mechanism for detachably coupling to the coupler.

27. The device of claim 15, further comprising:
a coupler for detachably coupling to the first end of the container, wherein the coupler comprises threads on an exterior surface for detachably coupling to a pump connector; and
a lid configured for detachably coupling to the coupler to thereby cover the coupler.

28. The device of claim 15, wherein the container has a volume capacity that is less than 40 ml.

29. A method for collecting and administering fluid from a breast, comprising:
detachably coupling a pump connector to a container, the pump connector being a component of a pump system, wherein the container has a first end with a first opening, and a second end with a second opening;
using the pump system to collect fluid from the breast; and
using the container to receive the fluid through the first opening at the first end.

30. The method of claim 29, further comprising:
removing the pump connector from the first end of the container after the fluid is collected from the breast; and
detachably coupling a plunger to the first end of the container.

31. The method of claim 30, further comprising using the plunger to deliver the fluid out of the container through the second opening at the second end of the container to a subject.
